(19) United States
(12) Patent Application Publication
(10) Pub. No.: US 2015/0038519 A1
(43) Pub. Date: Feb. 5, 2015

(54) ORGANIC COMPOUNDS

(71) Applicants: Sharon MATES, New York, NY (US); Lawrence WENNOGLE, New York, NY (US)

(72) Inventors: Sharon MATES, New York, NY (US); Lawrence WENNOGLE, New York, NY (US)

(21) Appl. No.: 14/516,382

(22) Filed: Oct. 16, 2014

Related U.S. Application Data
(63) Continuation of application No. 12/064,599, filed on Feb. 22, 2008.
(60) Provisional application No. 60/710,394, filed on Aug. 23, 2005.

(51) Int. Cl. C07D 487/04; A61K 31/519; A61K 45/06

(52) U.S. Cl. CPC C07D 487/04; A61K 45/06; A61K 31/519
USPC 514/262.1

(57) ABSTRACT

The invention provides a new method of treating diseases or conditions characterized by reduced dopamine D1 receptor signaling activity, such as Parkinson's disease, depression, and cognitive impairment of schizophrenia, comprising administering an effective amount of a 1,3,5,-substituted, 6,7-dihydro-1H-pyrazolo[4,3-d]pyrimidin-7-one to a patient in need thereof.

This application is a continuation application of U.S. patent application Ser. No. 12/064,599 filed Feb. 22, 2008, which is a 35 U.S.C. 371 National Phase Application of International Application No. PCT/US2006/033179, filed Aug. 23, 2006, which claims priority from U.S. Provisional Application No. 60/710,394, filed Aug. 23, 2005, the contents of each of which are hereby incorporated by reference.

### TECHNICAL FIELD

The present invention relates to a new use of 1,3,5,-substituted, 6,7-dihydro-1H-pyrazolo[4,3-d]pyrimidin-7-one compounds in the treatment of diseases involving PDE1-mediated suppression of the dopamine D1 receptor intracellular pathway, such as Parkinson's disease, depression and cognitive impairment associated with schizophrenia.

### BACKGROUND OF THE INVENTION

Eleven families of phosphodiesterases (PDEs) have been identified but only PDEs in Family I, the Ca²⁺-calmodulin-dependent phosphodiesterases (CaM-PDEs), have been shown to mediate the calcium and cyclic nucleotide (e.g. cAMP and cGMP) signaling pathways. The three known CaM-PDE genes, PDE1A, PDE1B, and PDE1C, are all expressed in central nervous system tissue. PDE1A is expressed throughout the brain with higher levels of expression in the CA1 to CA3 layers of the hippocampus and cerebellum and at a low level in the striatum. PDE1A is also expressed in the lung and heart. PDE1B is predominately expressed in the striatum, dentate gyrus, olfactory tract and cerebellum, and its expression correlates with brain regions having high levels of dopaminergic innervation. Although PDE1B is primarily expressed in the central nervous system, it may be detected in the heart. PDE1C is primarily expressed in olfactory epithelium, cerebellar granule cells, and striatum. PDE1C is also expressed in the heart and vascular smooth muscle.

Cyclic nucleotide phosphodiesterases decrease intracellular cAMP and cGMP signaling by hydrolyzing these cyclic nucleotides to their respective inactive 5′-monophosphates (5′AMP and 5′GMP). CaM-PDEs play a critical role in mediating signal transduction in brain cells, particularly within an area of the brain known as the basal ganglia or striatum. For example, NMDA-type glutamate receptor activation and/or dopamine D2 receptor activation result in increased intracellular calcium concentrations, leading to activation of effectors such as calmodulin-dependent kinase II (CaMKII) and calcineurin and to activation of CaM-PDEs, resulting in reduced cAMP and cGMP. Dopamine D1 receptor activation, on the other hand, leads to activation of calcium dependent nucleotide cyclases, resulting in increased cAMP and cGMP. These cyclic nucleotides in turn activate protein kinase A (PICA; cAMP-dependent protein kinase) and/or protein kinase G (PKG; cGMP-dependent protein kinase) that phosphorylate downstream signal transduction pathway elements such as DARPP-32 (dopamine and cAMP-regulated phosphoprotein) and cAMP responsive element binding protein (CREB).

CaM-PDEs can therefore affect dopamine-regulated and other intracellular signaling pathways in the basal ganglia (striatum), including but not limited to nitric oxide, noradrenergic, neurotensin, CCK, VIP, serotonin, glutamate (e.g., NMDA receptor, AMPA receptor), GABA, acetylcholine, adenosine (e.g., A2A receptor), cannabinoid receptor, natriuretic peptide (e.g., ANP, BNP, CNP) and endorphin intracellular signaling pathways.

Phosphodiesterase (PDE) activity, in particular, phosphodiesterase 1 (PDE1) activity, functions in brain tissue as a regulator of locomotor activity and learning and memory. PDE1 is a therapeutic target for regulation of intracellular signaling pathways, preferably in the nervous system, including but not limited to a dopamine D1 receptor, dopamine D2 receptor, nitric oxide, noradrenergic, neurotensin, CCK, VIP, serotonin, glutamate (e.g., NMDA receptor, AMPA receptor), GABA, acetylcholine, adenosine (e.g., A2A receptor), cannabinoid receptor, natriuretic peptide (e.g., ANP, BNP, CNP) or endorphin intracellular signaling pathway. For example, inhibition of PDE1B should act to potentiate the effect of a dopamine D1 agonist by protecting cGMP and cAMP from degradation, and similarly inhibit dopamine D2 receptor signaling pathways, by inhibiting PDE1 activity. Chronic elevation in intracellular calcium is linked to cell death in numerous disorders, particularly in neurodegenerative diseases such as Alzheimer's Parkinson's and Huntington's Diseases, and in disorders of the circulatory system leading to stroke and myocardial infarction. PDE1 inhibitors are therefore potentially useful in diseases characterized by reduced dopamine D1 receptor signaling activity, such as Parkinson's disease, restless leg syndrome, depression and cognitive impairment. See generally, WO 03/020702.

EP 0201188 and EP 0911333, the contents of which are incorporated herein by reference, disclose certain 1,3,5,-substituted, 6,7-dihydro-1H-pyrazolo[4,3-d]pyrimidin-7-one compounds, claimed to be useful for treatment of cardiovascular disease, erectile dysfunction, and other disorders. These compounds are not, however, taught or suggested to be useful for the treatment of diseases involving disorders of the dopamine D1 receptor intracellular pathway, particularly diseases such as Parkinson's disease, depression or cognitive impairment of schizophrenia.

### SUMMARY OF THE INVENTION

The invention provides a new method of treating diseases or conditions characterized by reduced dopamine D1 receptor signaling activity, such as Parkinson's disease, depression, and cognitive impairment of schizophrenia, all as described more fully below, comprising administering an effective amount of a 1,3,5,-substituted, 6,7-dihydro-1H-pyrazolo[4,3-d]pyrimidin-7-one, in free or pharmaceutically acceptable salt form (hereinafter a Compound of the Invention, e.g., as described below) to a patient in need thereof.

### DETAILED DESCRIPTION OF THE INVENTION

### Compounds for Use in the Methods of the Invention

Preferably, the Compounds of the Invention for use in the methods of treatment described herein are compounds of formula (1)

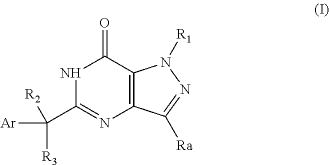

wherein

Rₐ is methyl or C₂-C₆ alkyl;

R₁ is H or C₁-C₄ alkyl;

each of R₂ and R₃ is independently selected from H and C₁-C₄ alkyl, or R₂ is H or C₁-C₄ alkyl and R₃ is OH, C₂-C₄ alkanoyloxy or fluoro, or R₂ and R₃ when taken together represent C₂-C₆ alkylene, or R₂ and R₃ when taken together with the carbon atom to which they are attached represent a carbonyl group;
Ar is either (a)

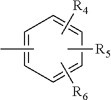

wherein

each of R₄, R₅ and R₆ is independently selected from
H
C₁-C₄ alkyl,
C₁-C₄ alkoxy,
C₁-C₄ alkoxy-Z—,
halo,
halo(C₁-C₄)alkyl,
phenoxy, optionally substituted by up to three substitutents
each of which substitutent is independently selected from halo,
C₁₋₄ alkyl, and C₁-C₄ alkoxy,
nitro,
hydroxy,
hydroxy-Z—,
C₂-C₄ alkanoyl,
amino,
amino-Z—,
(C₁-C₄ alkyl)NH,
(C₁-C₄ alkyl)₂N—,
(C₁-C₄ alkyl)NH—Z—,
(C₁-C₄ alkyl)₂N—Z—,
—COOH,
—Z—COOH,
—COO(C₁-C₄ alkyl),
—Z—COO(C₁-C₄ alkyl)
C₁-C₄ alkanesulphonamido,
C₁-C₄ alkanesulphonamido-Z—,
halo(C₁-C₄)alkanesulphonamido,
halo(C₁-C₄)alkanesulphonamido-Z—,
C₁-C₄ alkanamido,
C₁-C₄ alkanamido-Z—,
HOOC—Z—NH—,
HOOC—Z—NH—Z—,
(C₁-C₄ alkyl)OOC—Z—NH—,
(C₁-C₄ alkyl)OOC—Z—NH—Z—,
C₁-C₄ alkyl-NH—SO₂—NH—,
C₁-C₄ alkyl-NH—SO₂—NH—Z—,
(C₁-C₄ alkyl)₂—N—SO₂—NH—,
(C₁-C₄ alkyl)₂—N—SO₂—NH—Z—,
C₁-C₄ alkoxy CH═CH—Z—CONH—,
C₁-C₄ alkoxy CH═CHCONH
C₁-C₄ alkyl-SO₂—N(C₁-C₄ alkyl)-,
C₁-C₄ alkyl-SO₂—N(C₁-C₄ alkyl)-Z—,
(C₁-C₄ alkyl)NH—Z—SO₂—NH—,
(C₁-C₄ alkyl)₂N—Z—SO₂—NH—,
(C₁-C₄ alkyl)NH—Z—SO₂—NH—Z—,
(C₁-C₄ alkyl)₂N—Z—SO₂—NH—Z—,

benzenesulphonamido, optionally ring substituted by up to three substitutents each of which is independently selected from halo, C₁₋₄ alkyl, and C₁-C₄ alkoxy,
C₁-C₄ alkanoyl-N(C₁-C₄ alkyl)-,
C₁-C₄ alkanoyl-N(C₁-C₄ alkyl))-Z—,
C₁-C₄ alkoxycarbonyl-CH(CH₂OH)NHSO₂—,
—SO₃H,
—SO₂NH₂,
H₂NOC—CH(CH₂OH)—NHSO₂—,
HOOC—Z—O—, and
(C₁-C₄ alkyl)OOC—Z—O—,
   or optionally one of R₄, R₅ and R₆ is a G-Het group and wherein the others of R₄, R₅ and R₆ are independently selected from the R₄, R₅ and R₆ substitutents listed above;

Z is C₁-C₄ alkylene,

G is a direct link, Z, O, —SO₂NH—, SO₂, or —Z—N(C₁-C₄ alkyl)SO₂—,

Het is a 5- or 6-membered heterocyclic group containing 1, 2, 3 or 4 nitrogen heteroatoms; or 1 or 2 nitrogen heteroatoms and 1 sulphur heteroatom or 1 oxygen heteroatom; or the heterocyclic group is furanyl or thiophenyl; wherein the Het group is saturated or partially or fully unsaturated and optionally substituted by up to 3 substitutents, wherein each substitutent is independently selected from C₁-C₄ alkyl, oxo, hydroxy, halo, and halo(C₁-C₄) alkyl;

or (b) any one of the following bicyclic groups:
benzodioxolanyl,
benzodioxanyl,
benzimidazolyl,
quinolinyl,
indolyl,
quinazolinyl,
isoquinolinyl,
benzotriazolyl,
benzofuranyl,
benzothiophenyl,
quinoxalinyl, or
phthalizinyl,
   wherein said bicyclic Ar groups are linked to the neighbouring —C(R₂R₃)— group via the benzo ring portion,
   and wherein the heterocyclic portion of said bicyclic Ar group is optionally partially or fully saturated, said group being optionally substituted by one or more of C₁-C₄ alkyl, halo, hydroxy, oxo, amino, and C₁-C₄ alkoxy;

or a pharmaceutically acceptable salt of the compound, or a pharmaceutically acceptable solvate of the compound or the salt.

For example, Compounds of the Invention include 1,3,5,-substituted, 6,7-dihydro-1H-pyrazolo[4,3-d]pyrimidin-7-one, in free or pharmaceutically acceptable salt form, particularly compounds of Formula I or the following formulae:

1.2 Of Formula I wherein Rₐ is a C₂₋₅ alkyl group.

1.3 Of Formula I wherein Rₐ is a C₂₋₄ alkyl group.

1.4 Of Formula I wherein Rₐ is a C₃ alkyl group.

1.5 Of Formula I wherein Rₐ is methyl.

1.6 Of Formula I, 1.2, 1.3, 1.4 or 1.5 wherein R₁ is a C₁₋₆ alkyl group.

1.7 Of any of the preceding formulae wherein R₁ is a C₁₋₃ alkyl group.

1.8 Of any of the preceding formulae wherein R₁ is a methyl group.

1.9 Of any of the preceding formulae wherein R₂ is H.

1.10 Of any of the preceding formulae wherein R₃ is H.

1.11 Of any of the preceding formulae wherein R₄, R₅ and R₆ are independently selected from H₁ (C₁₋₄ alkyl)₂N—, C₁₋₄ alkanesulphonamido and benzenesulphonamido.

1.12 Of any of the preceding formulae wherein R₄, R₅ and R₆ are independently selected from H, diethylamino, methanesulphonamido and benzenesulphonamido.

1.13 Of any of the preceding formulae wherein Ar is 4-diethylaminophenyl.

1.14 Of any of the preceding formulae wherein Ar is 2-methanesulphonamidophenyl.

1.15 Of any of the preceding formulae wherein Ar is 4-benzenesulphonamidophenyl.

1.16 Of any of the preceding formulae wherein one of R₄, R₅ and R₆ is (C₁₋₄ alkyl)₂N— and wherein the other two of R₄, R₅ and R₆ are H.

1.17 Of any of the preceding formulae wherein one of R₄, R₅ and R₆ is diethylamino and wherein the other two of R₄, R₅ and R₆ are H.

1.18 Of any of the preceding formulae wherein Rₐ is methyl.

1.19 Of any of the preceding formulae wherein Rₐ is C₂-C₆ alkyl.

1.20 Of any of the preceding formulae wherein the compound is selected from the following:

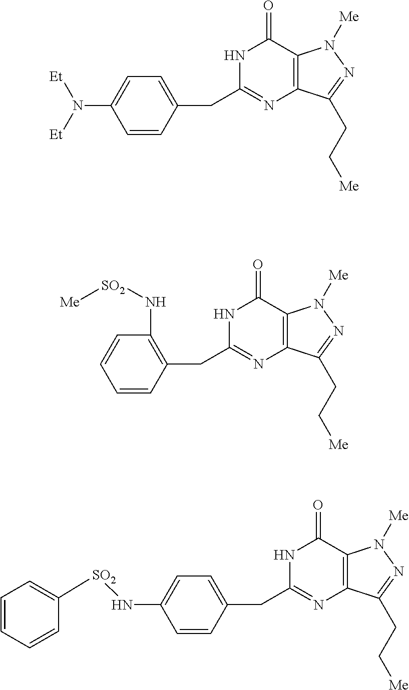 [SMILES: CCCc1nn(C)c2c(=O)[nH]c(Cc3ccc(N(CC)CC)cc3)nc12 ; CCCc1nn(C)c2c(=O)[nH]c(Cc3ccccc3NS(C)(=O)=O)nc12 ; CCCc1nn(C)c2c(=O)[nH]c(Cc3ccc(NS(=O)(=O)c4ccccc4)cc3)nc12]

1.21 Of any of the preceding formulae wherein the compound is

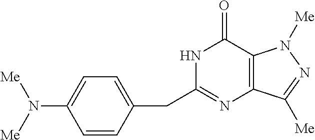 [SMILES: Cc1nn(C)c2c(=O)[nH]c(Cc3ccc(N(C)C)cc3)nc12]

1.22 A compound which is a 1,3,5,-substituted, 6,7-dihydro-1H-pyrazolo[4,3-d]pyrimidin-7-one, in free or pharmaceutically acceptable salt form, e.g. a compound of Formula I or according to any of formulae 1.2-1.21, wherein the compound inhibits phosphodiesterase-mediated (e.g., PDE1-mediated, especially PDE1B-mediated) hydrolysis of cGMP, e.g., with an IC₅₀ of less than 1 μM, preferably less than 25 nM in an immobilized-metal affinity particle reagent PDE assay, for example, as described in Example 1 below.

Compounds of the Invention may exist in free or salt form, e.g., as acid addition salts. In this specification unless otherwise indicated language such as Compounds of the Invention is to be understood as embracing the compounds in any form, for example free or acid addition salt form, or where the compounds contain acidic substituents, in base addition salt form. The Compounds of the Invention are intended for use as pharmaceuticals, therefore pharmaceutically acceptable salts are preferred. Salts which are unsuitable for pharmaceutical uses may be useful, for example, for the isolation or purification of free Compounds of the Invention or their pharmaceutically acceptable salts, are therefore also included.

Compounds of the Invention may in some cases also exist in prodrug form. For example when the compounds contain hydroxy or carboxy substituents, these substituents may form physiologically hydrolysable and acceptable esters. As used herein, “physiologically hydrolysable and acceptable ester” means esters of Compounds of the Invention which are hydrolysable under physiological conditions to yield acids (in the case of Compounds of the Invention which have hydroxy substituents) or alcohols (in the case of Compounds of the Invention which have carboxy substituents) which are themselves physiologically tolerable at doses to be administered. As will be appreciated the term thus embraces conventional pharmaceutical prodrug forms.

Methods of making and formulating the Compounds of the Invention, novel intermediates useful for making Compounds of the Invention, and methods of using the Compounds of the Invention for treatment of humans are disclosed in EP 0201188 and EP 0911333.

### Methods of Treatment Using Compounds of the Invention

The Compounds of the Invention are useful in the treatment of diseases characterized by disruption of or damage to cAMP and cGMP mediated pathways, e.g., as a result of increased expression of PDE1 or decreased expression of cAMP and cGMP due to inhibition or reduced levels of inducers of cyclic nucleotide synthesis, such as dopamine and nitric oxide (NO). By preventing the degradation of cAMP and cGMP by PDE1B, thereby increasing intracellular levels of cAMP and cGMP, the Compounds of the Invention potentiate the activity of cyclic nucleotide synthesis inducers.

The invention provides methods of treatment of any one or more of the following conditions:

(i) Neurodegenerative diseases involving suppression or dysfunction of the dopamine D1 receptor intracellular signaling pathway, including Parkinson's disease, restless leg syndrome, tremors, dyskinesias, Huntington's disease, Alzheimer's disease, and drug-induced movement disorders;

(ii) Mental disorders involving suppression or dysfunction of the dopamine D1 receptor intracellular signaling pathway, including depression, attention deficit disorder, attention deficit hyperactivity disorder, bipolar illness, anxiety, sleep disorders, cognitive impairment associated with schizophrenia, psychostimulant withdrawal, and drug addiction;

(iii) Any disease or disorder characterized by PDE1-mediated inhibition of the dopamine D1 receptor intracellular signaling pathway;

(iv) Circulatory and cardiovascular disorders, including cerebrovascular disease, stroke, congestive heart disease, hypertension, pulmonary hypertension, and sexual dysfunction;

(v) Respiratory and inflammatory disorders, including asthma, chronic obstructive pulmonary disease, and allergic rhinitis, as well as autoimmune and inflammatory diseases; and/or

(vi) Any disease or condition characterized by low levels of cAMP and/or cGMP (or inhibition of cAMP and/or cGMP signaling pathways) in cells expressing PDE1

comprising administering an effective amount of a Compound of the Invention, for example a Compound of Formula I or any of Formulae 1.2-1.22, to a human or animal patient, preferably a human, in need thereof.

The invention also provides a method for enhancing or potentiating dopamine D1 intracellular signaling activity in a cell or tissue comprising contacting said cell or tissue with an amount of a Compound of the Invention sufficient to inhibit PDE activity.

The invention also provides a method for treating a PDE1-related, especially PDE1B-related disorder, or a dopamine D1 receptor intracellular signaling pathway disorder, in a patient in need thereof comprising administering to the patient an effective amount of a Compound of the Invention that inhibits PDE1B, wherein PDE1B activity modulates phosphorylation of DARPP-32 and/or the G1uR1 AMPA receptor.

Compounds of the Invention may be used as a sole therapeutic agent, but may also be used in combination or for co-administration with other active agents. For example, as Compounds of the Invention potentiate the activity of D1 agonists, such as dopamine, they may be simultaneously, sequentially, or contemporaneously administered with conventional dopaminergic medications, such as levodopa and levodopa adjuncts (carbidopa, COMT inhibitors, MAO-B inhibitors), dopamine agonists, and anticholinergics, e.g., in the treatment of a patient having Parkinson's disease.

Thus, the invention further comprises a method of treating Parkinson's disease comprising administering simultaneously, sequentially, or contemporaneously administering therapeutically effective amounts of
(i) a Compound of the Invention, e.g., of Formula I or any of Formulae 1.2-1.22, and
(ii) a compound or compounds selected from
   dopaminergic agents, e.g., levodopa and levodopa adjuncts (carbidopa, COMT inhibitors, MAO-B inhibitors),
   dopamine agonists, and
   anticholinergics,
      to a patient in need thereof.

The present invention also provides
(i) a Compound of the Invention for use in the treatment of any disease or condition as hereinbefore set forth, or in a method of treatment as hereinbefore set forth;
(ii) the use of a Compound of the Invention in the manufacture of a medicament for treating a disease or condition as hereinbefore set forth, or manufacture of a medicament for use in a method of treatment as hereinbefore set forth; and
(iii) a pharmaceutical composition comprising a Compound of the Invention in combination or association with a pharmaceutically acceptable diluent or carrier for use in the treatment of a disease or condition as hereinbefore set forth, or for use in a method of treatment as hereinbefore set forth.

The words “treatment” and “treating” are to be understood accordingly as embracing prophylaxis and treatment or amelioration of symptoms of disease as well as treatment of the cause of the disease

Compounds of the Invention are in particular useful for the treatment of Parkinson's disease.

Dosages employed in practicing the present invention will of course vary depending, e.g. on the particular disease or condition to be treated, the particular Compound of the Invention used, the mode of administration, and the therapy desired. Compounds of the Invention may be administered by any suitable route, including orally, parenterally, transdermally, or by inhalation, but are preferably administered orally. In general, satisfactory results, e.g. for the treatment of diseases as hereinbefore set forth are indicated to be obtained on oral administration at dosages of the order from about 0.01 to 2.0 mg/kg. In larger mammals, for example humans, an indicated daily dosage for oral administration will accordingly be in the range of from about 0.75 to 150 mg, conveniently administered once, or in divided doses 2 to 4 times, daily or in sustained release form. Unit dosage forms for oral administration thus for example may comprise from about 0.2 to 75 or 150 mg, e.g. from about 0.2 or 2.0 to 50, 75 or 100 mg of a Compound of the Invention, together with a pharmaceutically acceptable diluent or carrier therefor.

Pharmaceutical compositions comprising Compounds of the Invention may be prepared using conventional diluents or excipients and techniques known in the galenic art. Thus oral dosage forms may include tablets, capsules, solutions, suspensions and the like.

### EXAMPLES

### 1. Measurement of PDE1B Inhibition In Vitro Using IMAP Phosphodiesterase Assay Kit

Phosphodiesterase 1B (PDE1B) is a calcium/calmodulin dependent phosphodiesterase enzyme that converts cyclic guanosine monophosphate (cGMP) to 5′-guanosine monophosphate (5′-GMP). PDE1B can also convert a modified cGMP substrate, such as the fluorescent molecule cGMP-fluorescein, to the corresponding GMP-fluorescein. The generation of GMP-fluorescein from cGMP-fluorescein can be quantitated, using, for example, the IMAP (Molecular Devices, Sunnyvale, Calif.) immobilized-metal affinity particle reagent.

Briefly, the IMAP reagent binds with high affinity to the free 5′-phosphate that is found in GMP-fluorescein and not in cGMP-fluorescein. The resulting GMP-fluorescein—IMAP complex is large relative to cGMP-fluorescein. Small fluorophores that are bound up in a large, slowly tumbling, complex can be distinguished from unbound fluorophores, because the photons emitted as they fluoresce retain the same polarity as the photons used to excite the fluorescence.

In the phosphodiesterase assay, cGMP-fluorescein, which cannot be bound to IMAP, and therefore retains little fluorescence polarization, is converted to GMP-fluorescein, which, when bound to IMAP, yields a large increase in fluorescence polarization (Amp). Inhibition of phosphodiesterase, therefore, is detected as a decrease in Amp.

### Enzyme Assay

Materials: All chemicals are available from Sigma-Aldrich (St. Louis, Mo.) except for IMAP reagents (reaction buffer, binding buffer, FL-GMP and IMAP beads), which are available from Molecular Devices (Sunnyvale, Calif.).

Assay: 3′,5′-cyclic-nucleotide-specific bovine brain phosphodiesterase (Sigma, St. Louis, Mo.) is reconstituted with 50% glycerol to 2.5 U/ml. One unit of enzyme will hydrolyze 1.0 μmole of 3′,5′-cAMP to 5′-AMP per min at pH 7.5 at 30° C. One part enzyme is added to 1999 parts reaction buffer (30 μM CaCl₂, 10 U/ml of calmodulin (Sigma P2277), 10 mM Tris-HCl pH 7.2, 10 mM MgCl₂, 0.1% BSA, 0.05% NaN₃) to yield a final concentration of 1.25 mU/ml. 99 μof diluted enzyme solution is added into each well in a flat bottom 96-well polystyrene plate to which 1 μl of test compound dissolved in 100% DMSO is added. The compounds are mixed and pre-incubated with the enzyme for 10 min at room temperature.

The FL-GMP conversion reaction is initiated by combining 4 parts enzyme and inhibitor mix with 1 part substrate solution (0.225 μM) in a 384-well microtiter plate. The reaction is incubated in dark at room temperature for 15 min. The reaction is halted by addition of 60 μl of binding reagent (1:400 dilution of IMAP beads in binding buffer supplemented with 1:1800 dilution of antifoam) to each well of the 384-well plate. The plate is incubated at room temperature for 1 hour to allow IMAP binding to proceed to completion, and then placed in an Envision multimode microplate reader (PerkinElmer, Shelton, Conn.) to measure the fluorescence polarization (Amp).

A decrease in GMP concentration, measured as decreased Amp, is indicative of inhibition of PDE activity. IC₅₀ values are determined by measuring enzyme activity in the presence of 8 to 16 concentrations of compound ranging from 0.0037 nM to 80,000 nM and then plotting drug concentration versus ΔmP, which allows IC₅₀ values to be estimated using nonlinear regression software (XLFit; IDBS, Cambridge, Mass.).

### 2. Depression

The Compounds of the Invention are evaluated in the following animal models for depression: the forced swim test as described in R. D. Porsolt, M. Le Pichon and M. Jalfre, Depression: a new animal model sensitive to antidepressant treatments, Nature (1977) 266: 730-732, and in the tail suspension test as described in R. Chermat, B. Thierry, J. A. Mico, L. Steru and P. Simon, Adaptation of the tail suspension test to the rat, J Pharmacol (1986) 17: 348-350.

### 3. Parkinson's Disease

The Compounds of the Invention are evaluated for their effect on the symptoms of Parkinson's disease using the unilateral 6-OHDA lesion model described in Ungerstedt, U., Stereotaxic mapping of the monoamine pathway in the rat brain. Acta Physiol. Scand. Suppl. (1971) 367: 1-48. This model provides a tool for investigating the pathophysiology of dopamine denervation. Animals with unilateral 6-OHDA dopamine denervation rotate ipsilaterally following administration of compounds which release dopamine, but contralaterally following administration of the dopamine precursor, L-DOPA, or dopaminergic agonists, such as apomorphine. Ungerstedt, U. and Arbuthnott, G. W., Quantitative recording of rotational behaviour in rats after 6-hydroxy-dopamine lesions of the nigrostriatal dopamine system. Brain Res. (1970) 24: 485-493. The latter effect has been attributed to a supersensitivity of dopamine receptors and/or their signal transduction mechanisms on the dopamine-depleted side (Ungerstedt, 1971). Contralateral rotation in this model, often referred to as Ungerstedt's model, is predictive for the anti-Parkinsonian action of a compound.

The Compounds of the Invention are further evaluated for their neuroprotective effect in the MTPT mouse model for Parkinson's Disease. Mice receiving 1-methyl-4-phenyl-1,2,3,6-tetrahydropridine (MPTP) suffer damage to the nigrostriatal dopaminergic pathway similar to that observed in Parkinson's Disease. The damage can be assessed by measuring the loss of tyrosine hydroxylase immunoreactivity (TH-IR) in the striatum and substantia nigra. The ability of the Compounds of the Invention to protect against such damage is evaluated using the method described in Murray, T. et al., LY503430, a Novel a-Amino-3-hydroxy-5-methylisoxazole-propionic Acid Receptor Potentiator with Functional, Neuroprotective and Neurotrophic Effects in Rodent Models of Parkinson's Disease, J.P.E.T. (2003) 306: 752-762.

1. A method of treating a disease or condition characterized by reduced dopamine D1 receptor signaling activity comprising administering an effective amount of a compound of the formula (1) 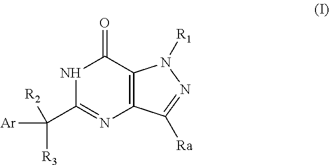
wherein
Rₐ is methyl or C₂-C₆ alkyl;
R₁ is H or C₁-C₄ alkyl;
each of R₂ and R₃ is independently selected from H and C₁-C₄ alkyl, or R₂ is H or C₁-C₄ alkyl and R₃ is OH, C₂-C₄ alkanoyloxy or fluoro, or R₂ and R₃ when taken together represent C₂-C₆ alkylene, or R₂ and R₃ when taken together with the carbon atom to which they are attached represent a carbonyl group;
Ar is either (a) 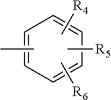
wherein
each of R₄, R₅ and R₆ is independently selected from
H
C₁-C₄ alkyl,
C₁-C₄ alkoxy,
C₁-C₄ alkoxy-Z—,
halo,
halo(C₁-C₄)alkyl,
phenoxy, optionally substituted by up to three substitutents each of which substitutent is independently selected from halo, C₁₋₄ alkyl, and C₁-C₄ alkoxy,
nitro,
hydroxy,
hydroxy-Z—,
C₂-C₄ alkanoyl,
amino,
amino-Z—,
(C₁-C₄ alkyl)NH,
(C₁-C₄ alkyl)₂N—,
(C₁-C₄ alkyl)NH—Z—,
(C₁-C₄ alkyl)₂N—Z—,
—COOH,
—Z—COOH,
—COO(C₁-C₄ alkyl),
—Z—COO(C₁-C₄ alkyl)
C₁-C₄ alkanesulphonamido,
C₁-C₄ alkanesulphonamido-Z—,
halo(C₁-C₄)alkanesulphonamido,
halo(C₁-C₄)alkanesulphonamido-Z—,
C₁-C₄ alkanamido,
C₁-C₄ alkanamido-Z—,
HOOC—Z—NH—,
HOOC—Z—NH—Z—,
(C₁-C₄ alkyl)OOC—Z—NH—,
(C₁-C₄ alkyl)OOC—Z—NH—Z—,
C₁-C₄ alkyl-NH—SO₂—NH—,
C₁-C₄ alkyl-NH—SO₂—NH—Z—,
(C₁-C₄ alkyl)₂—N—SO₂—NH—,
(C₁-C₄ alkyl)₂—N—SO₂—NH—Z—,
C₁-C₄ alkoxy CH═CH—Z—CONH—,
C₁-C₄ alkoxy CH═CHCONH
C₁-C₄ alkyl-SO₂—N(C₁-C₄ alkyl)-,
C₁-C₄ alkyl-SO₂—N(C₁-C₄ alkyl)-Z—,
(C₁-C₄ alkyl)NH—Z—SO₂—NH—,
(C₁-C₄ alkyl)₂N—Z—SO₂—NH—,
(C₁-C₄ alkyl)NH—Z—SO₂—NH—Z—,
(C₁-C₄ alkyl)₂N—Z—SO₂—NH—Z—,
benzenesulphonamido, optionally ring substituted by up to three substitutents each of which is independently selected from halo, C₁₋₄ alkyl, and C₁-C₄ alkoxy,
C₁-C₄ alkanoyl-N(C₁-C₄ alkyl)-,
C₁-C₄ alkanoyl-N(C₁-C₄ alkyl)-Z—,
C₁-C₄ alkoxycarbonyl-CH(CH₂OH)NHSO₂—,
—SO₃H,
—SO₂NH₂,
H₂NOC—CH(CH₂OH)—NHSO₂—,
HOOC—Z—O—, and
(C₁-C₄ alkyl)OOC—Z—O—,
or optionally one of R₄, R₅ and R₆ is a G-Het group and wherein the others of R₄, R₅ and R₆ are independently selected from the R₄, R₅ and R₆ substitutents listed above;
Z is C₁-C₄ alkylene,
G is a direct link, Z, O, —SO₂NH—, SO₂, or —Z—N(C₁-C₄ alkyl)SO₂—,
Het is a 5- or 6-membered heterocyclic group containing 1, 2, 3 or 4 nitrogen heteroatoms; or 1 or 2 nitrogen heteroatoms and 1 sulphur heteroatom or 1 oxygen heteroatom; or the heterocyclic group is furanyl or thiophenyl; wherein the Het group is saturated or partially or fully unsaturated and optionally substituted by up to 3 substitutents, wherein each substitutent is independently selected from C₁-C₄ alkyl, oxo, hydroxy, halo, and halo(C₁-C₄) alkyl;
or (b) any one of the following bicyclic groups:
benzodioxolanyl,
benzodioxanyl,
benzimidazolyl,
quinolinyl,
indolyl,
quinazolinyl,
isoquinolinyl,
benzotriazolyl,
benzofuranyl,
benzothiophenyl,
quinoxalinyl, or
phthalizinyl,
wherein said bicyclic Ar groups are linked to the neighbouring —C(R₂R₃)— group via the benzo ring portion,
and wherein the heterocyclic portion of said bicyclic Ar group is optionally partially or fully saturated, said group being optionally substituted by one or more of C₁-C₄ alkyl, halo, hydroxy, oxo, amino, and C₁-C₄ alkoxy;
in free form or in pharmaceutically acceptable salt form or in the form of a pharmaceutically acceptable solvate of the compound or the salt, to a human or animal patient in need thereof.

2. The method according to claim 1 wherein Rₐ is a C₂₋₅ alkyl group.

3. The method according to claim 1, wherein Rₐ is a C₂₋₄ alkyl group.

4. The method according to claim 1, wherein Rₐ is a C₃ alkyl group.

5. The method according to claim 1, wherein R₁ is a C₁₋₆ alkyl group.

6. The method according to claim 1, wherein R₁ is a C₁₋₃ alkyl group.

7. The method according to claim 1, wherein R₁ is a methyl group.

8. The method according to claim 1, wherein R₂ is H.

9. The method according to claim 1, wherein R₃ is H.

10. The method according to claim 1, wherein R₄, R₅ and R₆ are independently selected from H₁ (C₁₋₄ alkyl)₂N—, C₁₋₄ alkanesulphonamido and benzenesulphonamido.

11. The method according to claim 1, wherein R₄, R₅ and R₆ are independently selected from H, diethylamino, methanesulphonamido and benzenesulphonamido.

12. The method according to claim 1, wherein Ar is 4-diethylaminophenyl.

13. The method according to claim 1, wherein Ar is 2-methanesulphonamidophenyl.

14. The method according to claim 1, wherein Ar is 4-benzenesulphonamidophenyl.

15. The method according to claim 1, wherein one of R₄, R₅ and R₆ is (C₁₋₄ alkyl)₂N— and wherein the other two of R₄, R₅ and R₆ are H.

16. The method according to claim 1, wherein one of R₄, R₅ and R₆ is diethylamino and wherein the other two of R₄, R₅ and R₆ are H.

17. The method according to claim 1, wherein Rₐ is methyl.

18. The method according to claim 1, wherein Rₐ is C₂-C₆ alkyl.

19. The method according to claim 1, wherein the compound is selected from the following:

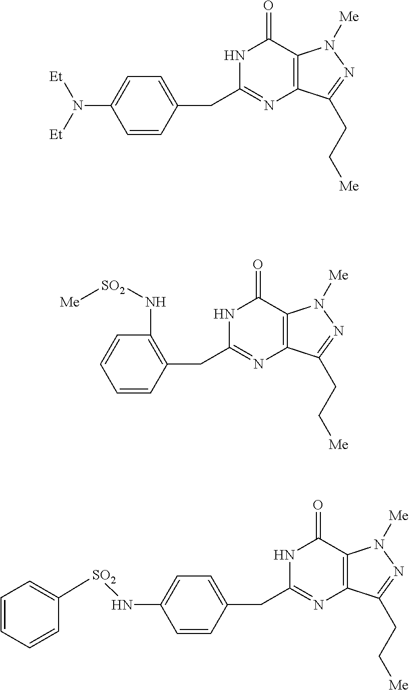 [SMILES: CCCc1nn(C)c2c(=O)[nH]c(Cc3ccc(N(CC)CC)cc3)nc12 ; CCCc1nn(C)c2c(=O)[nH]c(Cc3ccccc3NS(C)(=O)=O)nc12 ; CCCc1nn(C)c2c(=O)[nH]c(Cc3ccc(NS(=O)(=O)c4ccccc4)cc3)nc12]

20. The method according to claim 1, wherein the compound is

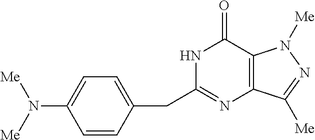 [SMILES: Cc1nn(C)c2c(=O)[nH]c(Cc3ccc(N(C)C)cc3)nc12]

21. The method according to claim 1, wherein the compound inhibits phosphodiesterase-mediated hydrolysis of cGMP.

22. A method of treating a disease or condition to be treated is selected from Parkinson's disease, restless leg, tremors, dyskinesias, drug-induced movement disorders, depression, attention deficit disorder, attention deficit hyperactivity disorder, bipolar illness, anxiety, sleep disorder, cognitive impairment associated with schizophrenia, psychostimulant withdrawal, and drug addiction, Huntington's disease, Alzheimer's disease, cerebrovascular disease, stroke, congestive heart disease, hypertension, pulmonary hypertension, sexual dysfunction, asthma, chronic obstructive pulmonary disease, allergic rhinitis, auto-immune and inflammatory diseases, comprising administering an effective amount of a compound of Formula I according to claim 1 in free or pharmaceutically acceptable salt or solvate form, to a human or animal patient in need thereof.

23. The method according to claim 22, wherein the disease or condition to be treated is Parkinson's disease.

24. The method according to claim 22, wherein the disease or condition to be treated is depression.

25. The method according to claim 22, wherein the disease or condition to be treated is cognitive impairment associated with schizophrenia.

26. The method according to claim 23 further comprising administering a compound or compounds selected from
dopaminergic agents,
dopamine agonists, and
anticholinergics,
to a patient in need thereof.

27. A method of treating a disease or condition characterized by PDE1-mediated inhibition of the dopamine D1 receptor intracellular signaling pathway, comprising administering an effective amount of a compound of Formula I according to claim 1, in free or pharmaceutically acceptable salt or solvate form, to a human or animal patient in need thereof.
